(19) United States
(12) Patent Application Publication
(10) Pub. No.: US 2024/0387002 A1
(43) Pub. Date: Nov. 21, 2024

(54) COMPUTATIONAL METHODS FOR PREDICTING ADHESION CHARACTERISTICS OF MOLECULAR COATINGS

(71) Applicant: Robert Bosch GmbH, Stuttgart (DE)

(72) Inventors: Georgy SAMSONIDZE, Cupertino, CA (US); Christoph SCHELLING, Stuttgart (DE); Karim GADELRAB, Boston, MA (US); Markus GANSER, Stuttgart (DE); Mordechai KORNBLUTH, Brighton, MA (US); Matthias KUEHNEL, Boeblingen (DE)

(21) Appl. No.: 18/197,509

(22) Filed: May 15, 2023

(51) Int. Cl. G16C 10/00; G16C 20/30

(52) U.S. Cl. CPC G16C 10/00; G16C 20/30; B81B 3/0005

(57) ABSTRACT

A computational method for predicting one or more adhesion characteristics of a candidate molecular coating. The computational method includes linking molecules of the candidate molecular coating to first anchor sites of a first substrate layer to obtain a first monolayer, arranging the first anchor sites in a two-dimensional (2D) lattice to obtain a close-packed first monolayer, spatially inverting the close-packed first monolayer to obtain a second monolayer associated with a second substrate layer, and predicting one or more adhesion characteristics of the candidate molecular coating for use in resisting stiction between the first and second substrate layers.

### TECHNICAL FIELD

This invention relates to computational methods for predicting adhesion characteristics of molecular coatings. The molecular coatings may be candidates for use in resisting stiction in micro electro mechanical systems (MEMS) inertial sensors.

### BACKGROUND

Inertial sensors have many applications. For instance, inertial sensors may be used in military and aerospace applications. The specific uses may include microgravity measurement, crash detection, smart ammunition, seat ejection triggers, and aircraft dynamics control. Inertial sensors may also be used in robotics (e.g., motion sensing to enable navigation), sports and fitness (e.g., tracking movements to measure performance), consumer electronics (e.g., screen orientation and gaming), and healthcare (e.g., monitoring movement in prosthetic limbs and rehabilitation).

One type of inertial sensor is a micro electro mechanical systems (MEMS) inertial sensor. MEMS inertial sensors are configured to transduce inertial forces into measurable electrical signals, which may be indicative of acceleration, inclination, and/or vibration of an object. MEMS inertial sensors may be classified into two main categories (i.e., accelerometers and gyroscopes). Accelerometers are configured to measure specific forces and/or accelerations. Gyroscopes are configured to measure angular velocities.

### SUMMARY

In one embodiment, a computational method for predicting one or more adhesion characteristics of a candidate molecular coating is disclosed. The computational method may be carried out using parameter-free quantum mechanics. The computational method may include linking molecules of the candidate molecular coating to first anchor sites of a first substrate layer to obtain a first monolayer, arranging the first anchor sites in a two-dimensional (2D) lattice to obtain a close-packed first monolayer, spatially inverting the close-packed first monolayer to obtain a second monolayer associated with a second substrate layer, and predicting one or more adhesion characteristics of the candidate molecular coating for use in resisting stiction between the first and second substrate layers. These steps may be performed for candidate molecular coatings and the method may identify one of the candidate molecular coatings for use in restricting stiction based on the relative values of the one or more adhesion characteristics of the candidate molecular coatings.

### BRIEF DESCRIPTION OF THE DRAWINGS

FIG. 1A is a schematic view of a conventional atomistic model of octadecyltrichlorosilane (ODTS) molecule monolayers coated onto the surfaces of first and second silica layers.

FIG. 1B depicts a graph including adhesion curves obtained from a conventional computational method using a classical force-field with about 25,000 atoms per simulation box.

FIG. 2 depicts a flowchart of a series of steps that may be carried out using density functional theory (DFT) software module to predict adhesion properties of different potential molecular coatings.

FIGS. 3A and 3B depict schematic views of an atomistic model of ODTS molecule monolayers coated onto silica surfaces according to a computational method of one embodiment (e.g., the embodiment shown in FIG. 2).

FIG. 3C depicts a graph plotting energy (eV/molecule) as a function of packing density (1/nm²) obtained using the computational method as described in FIG. 2.

FIG. 4A depicts a schematic view of an atomistic model of an interface between first and second ODTS monolayers coated onto silica surfaces according to the computational method as described in FIG. 2.

FIG. 4B depicts a graph including adhesion curves obtained using the computational method as described in FIG. 2.

FIG. 5 depicts a schematic diagram of a computing platform that may be utilized to implement DFT computational methodologies of one or more embodiments.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Except where otherwise expressly indicated, all numerical quantities in this description indicating amounts of material and/or use are to be understood as modified by the word “about” in describing the broadest scope of the invention. Practice within the numerical limits stated is generally preferred. Also, unless expressly stated to the contrary, term “polymer” includes “oligomer,” “ionomer,” “copolymer,” “terpolymer,” and the like; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed.

The first definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, the term “substantially,” “generally,” or “about” means that the amount or value in question may be the specific value designated or some other value in its neighborhood. These terms may be used to modify any numeric value disclosed or claimed herein. Generally, the term “about” denoting a certain value is intended to denote a range within ±5% of the value. As one example, the phrase “about 100” denotes a range of 100±5, i.e., the range from 95 to 105. Generally, when the term “about” is used, it can be expected that similar results or effects according to the invention can be obtained within a range of ±5% of the indicated value. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

It should also be appreciated that integer ranges explicitly include all intervening integers. For example, the integer range 1 to 10 explicitly includes 1, 2, 3, 4, 5, 6, 7, 8, 9, and 10. Similarly, the range 1 to 100 includes 1, 2, 3, 4, . . . 97, 98, 99, 100. Similarly, when any range is called for, intervening numbers that are increments of the difference between the upper limit and the lower limit divided by 10 can be taken as alternative upper or lower limits. For example, if the range is 1.1 to 2.1 the following numbers 1.2, 1.3, 1.4, 1.5, 1.6, 1.7, 1.8, 1.9, and 2.0 can be selected as lower or upper limits.

As used herein, the term “and/or” means that either all or only one of the elements of said group may be present. For example, “A and/or B” means “only A, or only B, or both A and B”. In the case of “only A”, the term also covers the possibility that B is absent, i.e., “only A, but not B”.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” as a subset.

The description of a group or class of materials as suitable for a given purpose in connection with one or more embodiments implies that mixtures of any two or more of the members of the group or class are suitable. Description of constituents in chemical terms refers to the constituents at the time of addition to any combination specified in the description and does not necessarily preclude chemical interactions among constituents of the mixture once mixed. First definition of an acronym or other abbreviation applies to all subsequent uses herein of the same abbreviation and applies mutatis mutandis to normal grammatical variations of the initially defined abbreviation. Unless expressly stated to the contrary, measurement of a property is determined by the same technique as previously or later referenced for the same property.

Developing better performing molecular coatings has proven difficult due to the difficulty of screening a large number of potential candidate molecules and a lack of data on the adhesion characteristics of each of the candidate molecules. While the adhesion properties of a molecule may be predicted using conventional atomistic modeling techniques, existing techniques have technical limitations that make them largely inapplicable to determine adhesion properties of molecular coatings.

A micro electro mechanical systems (MEMS) inertial sensor may include silicon surfaces that may experience in-use stiction. These silicon surfaces may be coated with self-assembled molecular coatings to resist or prevent in-use stiction. Self-assembled molecular coatings include thin films and layers that spontaneously arrange the molecules into a coating (or structure or pattern). The self-assembly may be a result of the properties of the molecules (e.g., size, shape, and/or chemistry). Non-limiting examples of self-assembled molecular coatings include self-assembled monolayers (SAMs) of organosilane molecules, which may otherwise be referred to as anti-stiction coatings (ASCs). Self-assembled molecular coatings are configured to lower surface free energies (SFE) and to reduce adhesive forces, thereby reducing the occurrence of the in-use stiction failure mode.

ASCs may be prone to degradation (1) during high temperature steps in the fabrication process, and (2) from repetitive mechanical shocks during operation of a MEMS inertial sensor or other device including an ASC. One possible mitigation strategy identifies a molecule for use as an ASC that would be more robust against degradation while having a reasonably low SFE. Due to the large number of potential candidate ASC molecules and the substantial experimental resources required for their hardware evaluation, some selection and/or prioritization of potential candidates is desirable. Such prioritization is obscured by the absence of data on the adhesion characteristics (e.g., SFE) of the potential candidates. What is needed is a computational method for predicting the adhesion characteristics of molecules that provides candidates for self-assembled molecular coating while not requiring excessive computational resources.

A current proposal for calculating SFEs of surfaces uses computational methods applying conventional atomistic modeling techniques. While these computational methods may be applied to bare crystalline surfaces, these computational methods are not suitable for SAMs due to a large system size (e.g., about 25,000 atoms) necessary to capture molecular disorder, which is essential for an accurate prediction of the SFE according to conventional atomistic techniques. Parameter-free quantum mechanical computational methods become prohibitively expensive and resource intensive for systems of that size. Moreover, classical force field computational methods are limited to a specific chemistry for which the force field parametrization is available (e.g., hydrocarbons on silica).

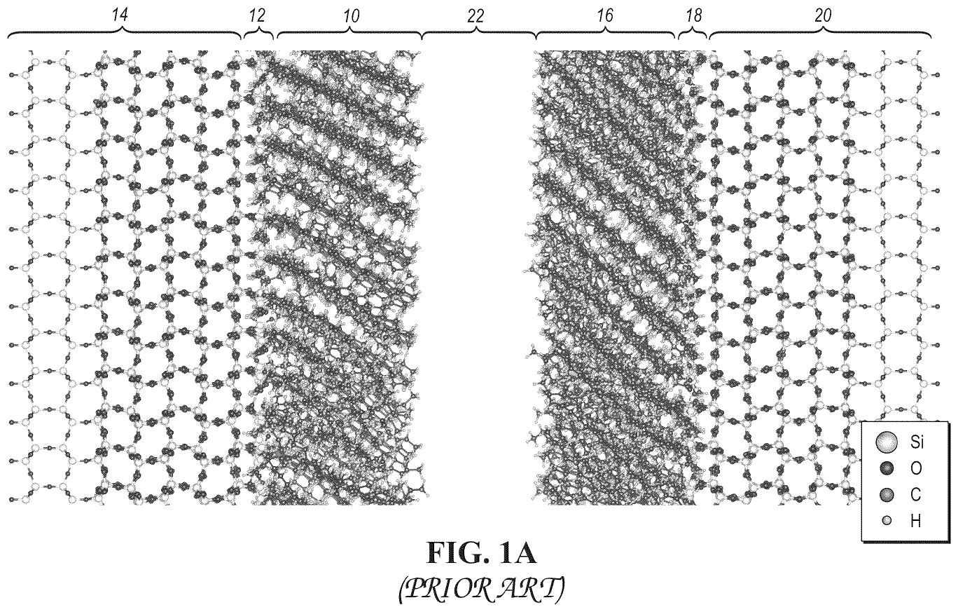

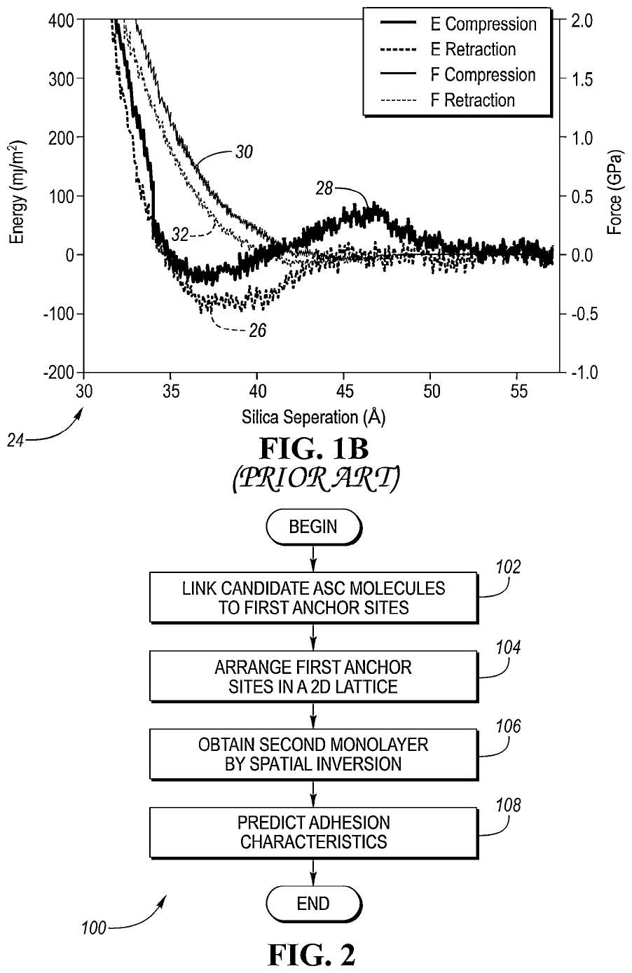

FIGS. 1A and 1B depicts an example using a computational method applying conventional atomistic modeling techniques. FIG. 1A is a schematic view of a conventional atomistic model of octadecyltrichlorosilane (ODTS) molecule monolayers coated onto the surfaces of silica layers. The silica layers may be included as part of a MEMS inertial sensor. As depicted in FIG. 1A, first ODTS molecule monolayer 10 is coated onto first surface 12 of first silica layer 14 and second ODTS molecule monolayer 16 is coated onto second surface 18 of second silica layer 20. As shown in FIG. 1A, gap 22 extends between first and second ODTS molecule monolayers 10 and 16. Using conventional computational method (e.g., molecular dynamics (MD) simulation using a classical force-field), first and second ODTS molecule monolayers 10 and 16 are brought together and then pulled apart to produce adhesion curves depicting net energy and force as a function of the intersurface separation distance (e.g., as depicted in FIG. 1B).

FIG. 1B depicts graph 24 including adhesion curves 26, 28, 30, and 32 obtained from a conventional computational method using a classical force-field with about 25,000 atoms per simulation box. In one or more embodiments, a simulation box refers to a virtual space configured to model the behavior of objects (e.g., particles, atoms, and/or molecules) by applying computational methods (e.g., numerical methods) to determine the motion of the objects. The simulation box may define the boundary conditions (e.g., size, shape, and/or periodicity) used in molecular dynamics simulations.

Graph 24 plots energy (mJ/m²) and force (GPa) as a function of silica separation (Å). Adhesion curve 26 plots energy (E) during compression as a function of silica separation (Å). Adhesion curve 28 plots energy (E) during retraction as a function of silica separation (Å). Adhesion curve 30 plots force (F) during compression as a function of silica separation (Å). Adhesion curve 32 plots force (F) during retraction as a function of silica separation (Å). The depths of the minima in the adhesion curves 26, 28, 30, and 32 give the surface free energy (SFE) and the maximum adhesion pressure (MAP).

The computations resulting in FIGS. 1A and 1B were performed using a large-scale atomic/molecular massively parallel simulator (LAMMPS) using an optimized potentials for liquid simulations-all atom (OPLS-AA) force field.

The computational method depicted in FIGS. 1A and 1B require a relatively large sized simulation box. What is needed is a computational method for predicting adhesion characteristics that uses a more manageably sized simulation box. This would permit the use of parameter-free quantum mechanical methods such as DFT instead of classical force-field methods which are limited to a specific chemistry fir which the force field parametrization is available.

In one or more embodiments, computational methods are disclosed that predict adhesion characteristics of potential molecular coatings using a manageable system size while relaxing or neglecting the impact of molecular disorder, thereby permitting the practical use of computational methods such as first-principles density functional theory (DFT). In one or more embodiments, the computational methods are utilized to enable computational screening of molecular chemistries for potential use as ASC molecules in connection with resisting or restricting stiction in MEMS inertial sensors.

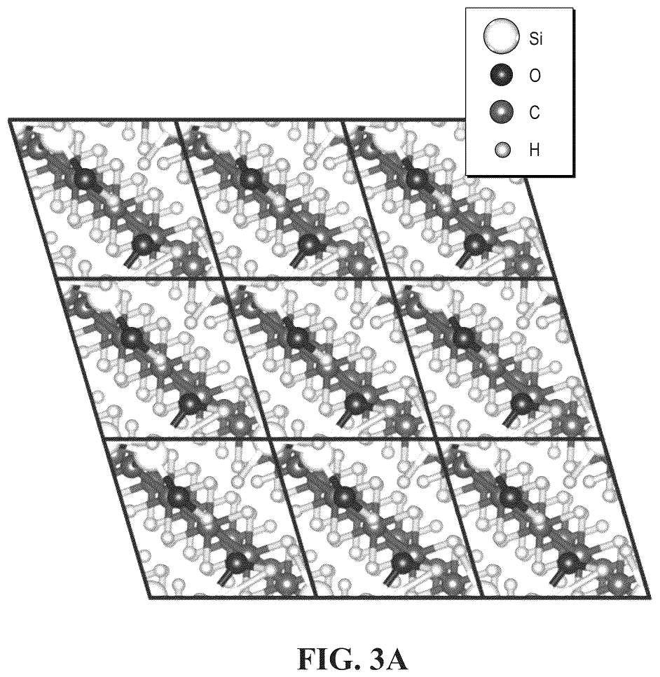

FIG. 2 depicts flowchart 100 of a series of steps carried out by a computational method using DFT to predict the adhesion characteristics of a candidate molecular coating. The candidate molecular coating may be a candidate ASC molecule. In one or more embodiments, one or more of the steps may be omitted, rearranged and/or augmented.

Step 102 of flowchart 100 is directed to linking a candidate ASC molecule to first anchor sites. The first anchor sites may be represented as molecules on a surface of a MEMS inertial sensor. The surface may be a silica surface and the first anchor sites may be silanol molecules. In one or more embodiments, the result of step 102 is the virtual formation of a first monolayer of a first surface of a first substrate layer which represents a coated silica surface.

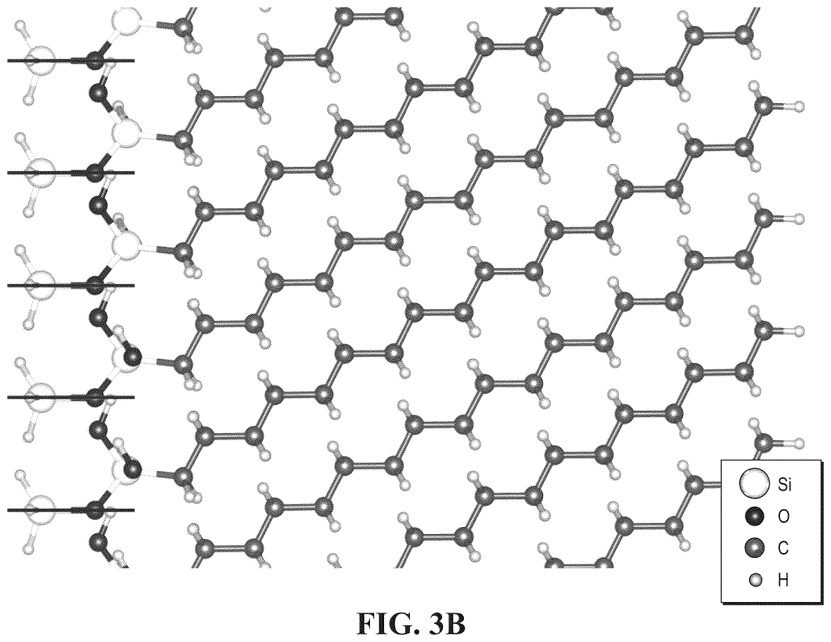

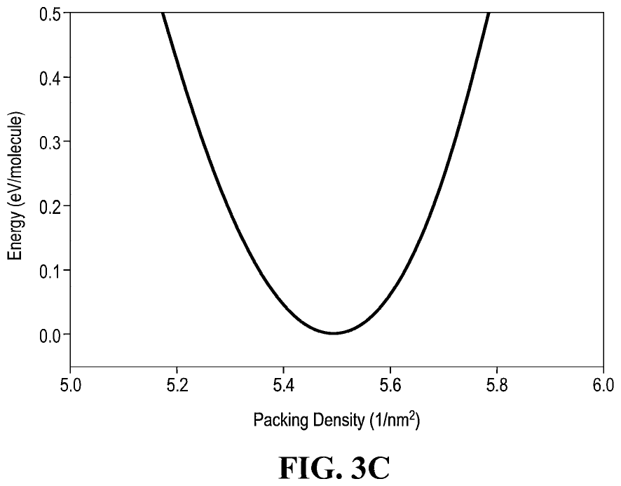

Step 104 of flowchart 100 is directed to arranging the first anchor sites in a two-dimensional (2D) lattice. This arrangement may be made so that the candidate ASC molecules in the first monolayer are pointing in the same direction. The vectors of the 2D lattice may be obtained by constructing a minimal enclosing parallelogram (MEP) of the atomic positions of the molecules of the candidate molecular coating projected onto a 2D plane. The atomic positions and the 2D lattice vectors may be optimized by minimizing DFT energy while constraining the direction of the bonds at the first anchor sites to be normal the 2D plane. In FIG. 3B, the bonds at the first anchor sites are silicon-oxygen bonds. In one or more embodiments, step 104 produces a close-packed monolayer with perfect defect-free packing of molecules.

Step 106 of flowchart 100 is directed to obtaining a second monolayer by spatial inversion of the first monolayer. The spatial inversion may involve transforming the molecules' position and momentum coordinates. In one or more embodiments, the first and second monolayers are placed parallel to each other such that the molecules face inward (e.g., toward each other) and the anchor groups face outward (e.g., away from the molecules of the first and second monolayers). The first and second monolayers may be shifted laterally with respect to each other. The lateral shift may be optimized to maximize a shortest interatomic distance from any atom in the first monolayer to any atom in the second monolayer, or vice versa. The resulting arrangement provides alignment of the peaks of one of the monolayers and the valleys of the other monolayer or vice versa, such that the first and second monolayers may be brought close to each other as spatially possible. This aligned arrangement may also provide a minimal change in the molecular conformation when the first and second monolayers are brought together, thereby resisting or minimizing conformational changes in the molecules.

Step 108 of flowchart 100 includes predicting adhesion characteristics of the candidate molecular coating. The adhesion characteristics may be net energy and force as a function of a distance between the first and second monolayers, while atoms within each of the first and second monolayers are kept fixed with respect to each other. In one or more embodiments, relaxation of atoms within monolayers is not performed to prevent any changes in the molecular conformation as the first and second monolayers approach each other. Changes in molecular conformation may lead to large errors in the predicted SFE and MAP.

The computational method described in FIG. 2 may be performed using a significantly reduced system size over the conventional computational method disclosed herein. For instance, while the conventional computational method used a large system size to capture molecular disorder (about 250 molecules and two silica surfaces resulting in about 25,000 atoms per simulation box), the computational methods of one or more embodiments significantly reduces system size (e.g., two molecules or about 100 atoms per simulation box) by relaxing or neglecting the impact of molecular disorder, thereby enabling DFT calculations to identity adhesion characteristics of different candidate coating molecules.

FIGS. 3A and 3B depict schematic views of an atomistic model of ODTS molecule monolayers coated onto silica surfaces according to one embodiment as described in FIG. 2. FIG. 3C depicts a graph plotting energy (eV/molecule) as a function of packing density (1/nm²) obtained using the computational method described in FIG. 2. The computational method used to generate FIG. 3C uses DFT and about 50 atoms per simulation box.

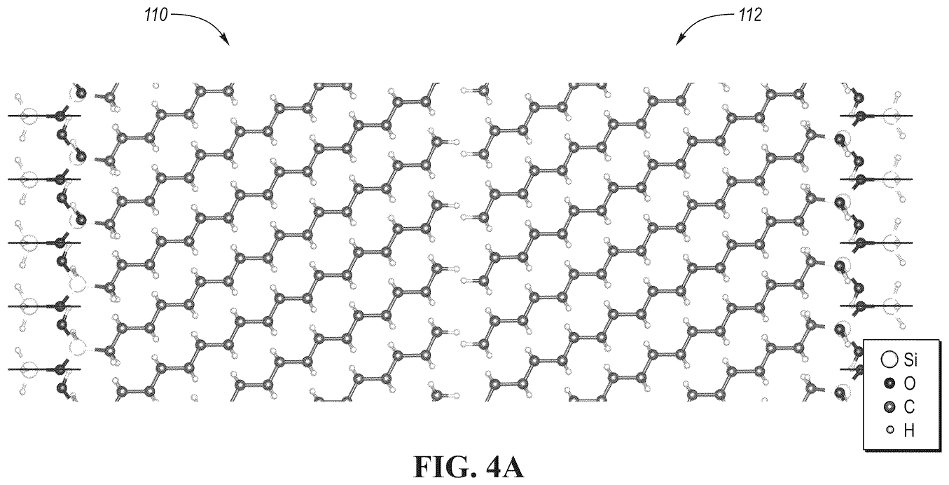

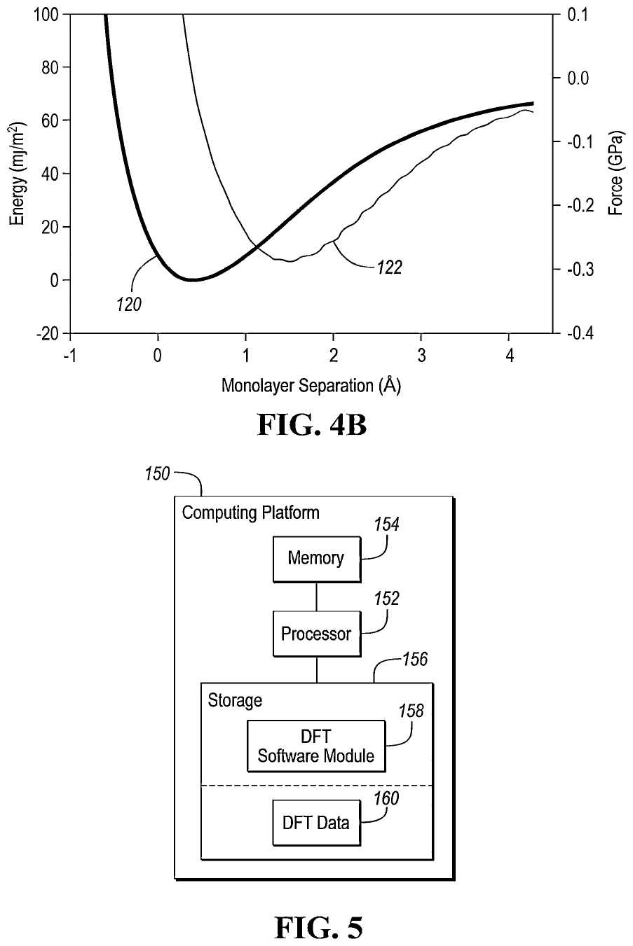

FIG. 4A depicts a schematic view of an atomistic model of an interface between first and second ODTS monolayers 110 and 112 coated onto silica surfaces according to one embodiment as described in FIG. 2. FIG. 4B depicts a graph including adhesion curves obtained from a computational method of one embodiment as described in FIG. 2. The graph plots energy (mJ/m²) (curve 120) and force (GPa) (curve 122) as a function of monolayer separation (Å). The black lines in FIGS. 3B and 4A are the constrained bond direction described above with respect to step 104.

The computations resulting in FIGS. 3A through 3C and FIGS. 4A and 4B may be performed using CP2K with Perdew-Burke-Ernzerhof (PBE) exchange-correlation function and DFT-D3 dispersion correction and triple-zeta split-valence double-polarized polarization-consistent (TZV2PX) basis set. CP2K is a computational algorithm that performs quantum chemical calculations and molecular simulations on molecular systems. CP2K uses DFT to describe the electronic structure of molecules and materials. The PBE exchange-correlation functional is used in DFT calculations to obtain electronic structures of molecules. DFT-D3 dispersion correction is a dispersion correction method used to improve DFT calculation accuracy. The TZV2PX basis set is a triple-zeta split-valence double-polarized polarization-consistent Gaussian basis set configured to be used with DFT calculations.

Table 1 lists the adhesion properties of a self-assembled monolayer of ODTS molecules with the chemical formula C₁₈H₃₇SiCl₃.

TABLE 1

| | Packing Density | Surface Free | Maximum Adhesion |
|---|---|---|---|
| Method | (1/nm²) | Energy (mJ/m²) | Pressure (GPa) |
| Experimental | — | 22 | — |
| Conventional | 2.7 | 41 | 0.04 |
| Disclosed | 5.5 | 75 | 0.29 |
| Embodiment | | | |

As shown in Table 1, the computational methods of one or more embodiments may overestimate experimental SFE values. The overestimation may result from relaxing or neglecting molecular disorder and finite temperature effect. In one or more embodiments, the computational method provides an upper bound on the SFE to compensate for relaxing or neglecting the impact of molecular disorder, which tends to lower the SFE. While the absolute values of SFE may be overestimated, the computational methods of one or more embodiments capture trends when comparing different coating molecules candidates, and thus may be used for selection and/or prioritization of the candidates.

The computational methods of one or more embodiments may also be used to predict the optimal packing density of molecules, thereby excluding bias due to a fixed density of binding sites on the simulated surface, which may be inherent in the conventional computational method.

The computational methods of one or more embodiments are not limited in their application to surfaces of MEMS inertial sensor and may be applied to other applications involving SAMs. Non-limiting examples of these applications includes nanoelectromechanical systems (NEMS), microfabrication, nanofabrication, water repellency, surface wetting, surface passivation, corrosion resistance, electrode coatings for electrochemistry and electronics, SAM nanoparticle arrays, molecular electronics, and biotechnology.

The computational methods using DFT of one or more embodiments are implemented using a computing platform, such as the computing platform 150 illustrated in FIG. 5. The computing platform 150 may include a processor 152, memory 154, and non-volatile storage 156. The processor 152 may include one or more devices selected from high-performance computing (HPC) systems including high-performance cores, microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory 154. The memory 154 may include a single memory device or a number of memory devices including, but not limited to, random access memory (RAM), volatile memory, non-volatile memory, static random access memory (SRAM), dynamic random access memory (DRAM), flash memory, cache memory, or any other device capable of storing information. The non-volatile storage 156 may include one or more persistent data storage devices such as a hard drive, optical drive, tape drive, non-volatile solid state device, cloud storage or any other device capable of persistently storing information.

Processor 152 may be configured to read into memory 154 and execute computer-executable instructions residing in DFT software module 158 of the non-volatile storage 156 and embodying DFT algorithms and/or methodologies of one or more embodiments. Software module 158 may include operating systems and applications. Software module 158 may be compiled or interpreted from computer programs created using a variety of programming languages and/or technologies, including, without limitation, and either alone or in combination, Java, C, C++, C#, Objective C, Fortran, Pascal, Java Script, Python, Perl, and PL/SQL.

Upon execution by the processor 152, the computer-executable instructions of the DFT software module 158 may cause the computing platform 150 to implement one or more of the DFT algorithms and/or methodologies disclosed herein. Non-volatile storage 156 may also include DFT data 160 supporting the functions, features, calculations, and processes of the one or more embodiments described herein.

The program code embodying the algorithms and/or methodologies described herein is capable of being individually or collectively distributed as a program product in a variety of different forms. The program code may be distributed using a computer readable storage medium having computer readable program instructions thereon for causing a processor to carry out aspects of one or more embodiments. Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions, acts, and/or operations specified in the flowcharts or diagrams. In certain alternative embodiments, the functions, acts, and/or operations specified in the flowcharts and diagrams may be re-ordered, processed serially, and/or processed concurrently consistent with one or more embodiments. Moreover, any of the flowcharts and/or diagrams may include more or fewer nodes or blocks than those illustrated consistent with one or more embodiments.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A computational method for predicting one or more adhesion characteristics of a candidate molecular coating, the method comprising:
linking molecules of the candidate molecular coating to first anchor sites of a first substrate layer to obtain a first monolayer;
arranging the first anchor sites in a two-dimensional (2D) lattice to obtain a close-packed first monolayer;
spatially inverting the close-packed first monolayer to obtain a second monolayer associated with a second substrate layer; and
predicting one or more adhesion characteristics of the candidate molecular coating for use in resisting stiction between the first and second substrate layers.

2. The computational method of claim 1, wherein the arranging step includes constructing a minimal enclosing parallelogram (MEP) of atomic positions of the molecules of the candidate molecular coating projected onto a 2D plane to obtain lattice vectors of the 2D lattice.

3. The computational method of claim 2, wherein the arranging step includes constraining a direction of bonds at the first anchor sites to be normal to the 2D plane.

4. The computational method of claim 3, wherein the candidate molecular coating includes organosilane molecules.

5. The computational method of claim 1, wherein the one or more adhesion characteristics includes maximum surface free energy (SFE) or adhesion pressure (MAP).

6. The computational method of claim 1, wherein the spatially inverting step includes placing the molecules of the first monolayer and the second monolayer parallel to each other.

7. The computational method of claim 1, wherein the spatially inverting step includes laterally shifting the first monolayer and the second monolayer with respect to each other.

8. The computational method of claim 7, wherein the laterally shifting step includes maximizing a shortest interatomic distance from an atom in the first monolayer to an atom in the second monolayer.

9. The computational method of claim 8, wherein the peaks of the first monolayer are aligned with the valleys of the second monolayer and vice versa to minimize the gaps between the two monolayers.

10. The computational method of claim 9, wherein the aligned arrangement is configured to provide a minimal change in a molecular conformation.

11. The computational method of claim 1, wherein the one or more adhesion characteristics includes net energy and/or net force as a function of a distance between the first monolayer and the second monolayer.

12. The computational method of claim 1, wherein the one or more adhesion characteristics are surface free energy (SFE) and maximum adhesion pressure (MAP).

13. The computational method of claim 12, wherein the one or more adhesion characteristics includes SFE, and further comprising bounding an upper value of the SFE.

14. The computational method of claim 1, wherein the candidate molecular coating is a self-assembled molecular coating.

15. The computational method of claim 1, wherein the linking, arranging, spatially inverting, and predicting steps are performed using density functional theory (DFT) calculations.

16. The computational method of claim 1, wherein the linking, arranging, spatially inverting, and predicting steps are performed on a system size of 100 atoms or less per simulation box.

17. The computational method of claim 1, wherein the linking, arranging, spatially inverting, and predicting steps are performed while relaxing or neglecting an impact from molecular disorder of the molecules.

18. The computational method of claim 1, wherein the candidate molecular coating includes organosilane molecules.

19. A computational method for predicting one or more adhesion characteristics of candidate molecular coatings, the comprising:
for each of the candidate molecular coatings, performing the following steps:
linking first molecules of the candidate molecular coating to first anchor sites of a first substrate layer to obtain a first monolayer;
arranging the first anchor sites in a two-dimensional (2D) lattice to obtain a close-packed first monolayer;
spatially inverting the close-packed first monolayer to obtain a second monolayer associated with a second substrate layer; and
predicting one or more adhesion characteristics of the candidate molecular coating; and
identifying one of the candidate molecular coatings for use in restricting stiction based on relative values of the one or more adhesion characteristics of the candidate molecular coatings.

20. A computational method using parameter-free quantum mechanics to predict one or more adhesion characteristics of a candidate molecular coating, the method comprising:
linking molecules of the candidate molecular coating to first anchor sites of a first substrate layer to obtain a first monolayer;
arranging the first anchor sites in a two-dimensional (2D) lattice to obtain a close-packed first monolayer;
spatially inverting the close-packed first monolayer to obtain a second monolayer associated with a second substrate layer; and
predicting one or more adhesion characteristics of the candidate molecular coating for use in resisting stiction between the first and second substrate layers.
